(19) United States
(12) Patent Application Publication
(10) Pub. No.: US 2004/0208919 A1
(43) Pub. Date: Oct. 21, 2004

(54) Vaccination against prion diseases

(76) Inventors: Yves C. Nicolau, Newton, MA (US); Ernest Kapetanovic, Woburn, MA (US)

Correspondence Address: Samir Yastas, diagenic, dusseldorf, germany, 40212, DE

(21) Appl. No.: 10/461,864

(22) Filed: Jun. 13, 2003

Related U.S. Application Data
(60) Provisional application No. 60/388,795, filed on Jun. 13, 2002.

(51) Int. Cl. A61K 39/00; A61K 9/127

(52) U.S. Cl. 424/450; 424/185.1

(57) ABSTRACT

Compositions and methods for the treatment or prevention of neurodegenerative diseases caused by the accumulation of prions. Therapeutic vaccines, antisera and molecular constructs are described. The vaccine is composed of an antigen, such as a prion peptide fragment or epitope that is preferably provided in a liposomal bilayer. In a preferred embodiment, the antigen is a modified amyloid peptide, preferably a palmitoylated PrP^{c}₁₀₆₋₁₂₆ peptide. Preferably, the antigen is administered in a liposomal bilayer. When administered to an animal, the vaccine elicits a local or systemic, immunogen-specific immune response against amyloid proteins, peptides or fragments, and prevents, stops or hinders amyloid deposition caused by prions.

### CROSS-REFERENCE TO RELATED APPLICATIONS

The present application claims benefit of U.S. Provisional Application No. 60/388,795 filed Jun. 13, 2002.

### FIELD OF INVENTION

The present invention is directed to methods and compositions for the prevention and treatment of neurodegenerative diseases caused by accumulation of prions.

### BACKGROUND

Prions are infectious pathogens that cause central nervous system spongiform encephalopothies such as scrapie in sheep, transmissible mink encephalopathy, chronic wasting disease in muledeer and elk, bovine spongiform encephalopathy in cattle and Creutzfeldt-Jacob disease, Gerstmann-Strussler-Scheinker syndrome, fatal familial insomnia, kuru and alpers syndrome in humans.

These diseases are characterized by loss of motor control, dementia, paralysis, wasting and eventually death. Humans are infected either through an infectious agent or through hereditary transmission. While not wishing to be bound, it is currently theorized that disease causing prions are modified forms of a normal cellular protein known as PrP^{c}. The modified form of PrP^{c} which is believed to cause disease is PrP^{Sc} (scrapie).

Although prions multiply, there is no evidence that they contain nucleic acid. PrP_{Sc} is derived from the non-infectious, cellular protein PrP^{c} by a posttranslational process during which PrP^{c} undergoes a three stage transition as follows. The normal cellular isoform of prion protein (PrP^{c}) progresses to the infectious form (scrapie isoform of prion protein or PrP^{Sc}), which progresses to prion protein PrP27-30 (Gajdusek, D. C. (1988) Mt. Sinai J. Med. 55:3-5; Prusiner, S. B. (1992) Biochemistry 31:12277-12288. The term PrP^{Sc}, as used herein, refers not only to the specific prion protein identified in sheep, but also to those homologous proteins found in many other species.

PrP^{Sc} is almost identical to the natural form of the protein PrP^{c}. The cellular, non-toxic prion protein (PrP^{c}) is a sialoglycoprotein having a molecular weight from 33 to 35K, which is expressed predominantly in neurons (Oeseh, B., et al. (1985) Cell 40:735-746; Chesebro, B., et al. (1985) Nature 315:331-333; Kretzsclunar, H. A., et al., (1986) Amer. J. Pathol. 122:1-5). In the diseases mentioned above, PrP^{c} is converted into an altered form (PrP^{Sc}) which is distinguishable from its normal homologue by its relative resistance to protease digestion (Bolton, D. C., et al. (1982) Science 218:1309-1311; McKinley, M. P., et al. (1983) Cell 35:57-62; Forloni, G., et al. (1993) Nature 362:543-546). PrP^{Sc} accumulates in the central nervous system of affected animals and individuals and its protease-resistant core aggregates extracellarly (Prusiner, S. B. et al. (1983) Cell 35:349-358; Tagliavini, F., et al. (1991) EMBO J. 10:513-519). Currently there is no available vaccine or treatment for prion diseases.

Conventional vaccines generally include either purified antigens or an attenuated form of a pathogen that can be administered to a patient to generate an immune response. Problems exist with the application of traditional methods of vaccine production for prion associated diseases because it is believed that it is the molecular structure of the PrP^{Sc} protein itself rather than the nucleic acid sequence which passes on the infectivity. Traditional methods of viral vaccine production involve the inactivation of the virus through techniques such as heat treatment or serial passaging of the virus through a culture. However, these approaches would lead to a conformational change in the protein and a loss of the antigenic epitopic sites in the infectious prions. There is therefore a need to obtain antigenic but non-infective prion proteins which can be used in vaccines.

### SUMMARY

Compositions and methods for the treatment and prevention of prion accumulation and prion related diseases are described herein. Synthetic prion peptides were created to determine antigenic fragments that are useful in the preparation of a vaccine. When tested in rat hippocampal cultures, the synthetic peptides demonstrated that neuronal death occurs from chronic exposure to micromolar concentrations of prion protein fragments, particularly PrP^{c}₁₀₆₋₁₂₆.

Also described herein is a vaccine and method for administering a vaccine that elicits a local or systemic, immunogen-specific immune response against amyloid proteins, peptides or fragments, and prevents, stops or hinders amyloid deposition caused by prions. The vaccine is composed of an antigen, such as a prion peptide fragment or epitope that is preferably provided in a liposomal bilayer. In a preferred embodiment, the antigen is composed of a modified amyloid peptide, preferably palmitoylated PrP^{c}₁₀₆₋₁₂₆ peptide. Preferably, the antigen is administered in a liposomal bilayer.

A pharmaceutical composition, or vaccine composition, in an amount effective to prevent, stop or impede one or more symptoms of a disease state involving abnormal accumulation or molecular organization of prion protein, assemblies, fibrils, filaments, tangles, or amyloid deposits is described. The vaccine composition includes an antigen to stimulate production of anti-prion antibodies having the ability to dissolve accumulated prions. Also provided is a method for treating a disease state associated with neurodegenerative prion by the administration of a pharmaceutically effective amount of the vaccine composition to a patient. The composition may be administered by any means including, but not limited to, intravenously, intraperitoneally, subcutaneously, intradermally, intramuscularly, transdermally, intraarticularly, intracranially or intraspinally.

Methods for making compositions containing antisera raised to the epitope of the vaccine compositions described herein are also provided. These antisera compositions are capable of dissolving prion aggregates containing the epitope or antigen, such as, but not limited to PrP^{c}₁₀₆₋₁₂₆. The antisera compositions are effective in the treatment or prevention of the accumulation of amyloid protein or the formation of prion assemblies, fibrils, filaments, tangles, plaques or amyloid deposits and, therefore, disease states associated with neurodegenerative prion diseases.

Also described herein are methods and compositions for preventing or alleviating the symptoms of disease states associated with the accumulation or molecular organization of prion proteins or prion plaques by the administration of a pharmaceutically effective amount of antisera to a patient.

The methods provided herein are particularly useful in the treatment of prion associated diseases such as central nervous system spongiform encephalopothies such as scrapie in sheep, transmissible mink encephalopathy, chronic wasting disease in muledeer and elk, bovine spongiform encephalopathy in cattle and Creutzfeldt-Jacob disease, Gerstmann-Strussler-Scheinker syndrome, fatal familial insomnia, kuru, and Alpers syndrome in humans.

These and other objects, features, and advantages of the present invention will become apparent after a review of the following detailed description of the disclosed embodiments and the appended claims.

### BRIEF DESCRIPTION OF THE FIGURES

FIG. 1 is a cartoon showing a synthetic strategy for anchoring lipopeptides in liposomes.

### DETAILED DESCRIPTION

Methods and compositions are provided herein for the treatment and prevention of neurodegenerative diseases caused by the accumulation of prions into amyloid plaques, such as scrapie in sheep, transmissible mink encephalopathy, chronic wasting disease in muledeer and elk, bovine spongiform encephalopathy in cattle and Creutzfeldt-Jacob disease, Gerstmann-Strussler-Scheinker syndrome, fatal familial insomnia, kuru, and Alpers syndrome in humans.

The cellular, non-toxic prion protein PrP^{c} is a sialoglycoprotein of molecular weight 33-35K that is expressed predominantly in neurons. It is either bound to the cell surface by a glycosyl phosphatidylinositol anchor or secreted as a soluble derivative. The non-toxic protein, PrP^{c}, is converted to the toxic form PrP^{Sc}, which accumulates and forms amyloid fibrils in the central nervous system of affected animals. PrP^{Sc} is distinguishable from its normal homologue by its relative resistance to protease digestion.

A possible mechanism of neurotoxicity was investigated in a model system aimed at detecting and analyzing ionic channel formations upon the interaction of peptides or proteins with lipid bilayers. Low pH, which favors channel formation by PrP₁₀₆₋₁₂₆ converts this peptide from an α-helical to β-sheet conformation. Peptide mapping of PrP^{Sc} with Edman sequencing and mass spectrometry revealed no differences between its amino acid sequence and that predicted from the PrP^{c} gene sequence, and no chemical modifications were found that might distinguish PrP^{Sc} from PrP^{c}. However, Fourier Transform infrared spectroscopy and circular dichroism spectroscopy revealed a significant conformational difference between PrP^{Sc} and PrP^{c}.

PrP^{c} is essentially α-helical with little or no β-sheet confirmation, whereas PrP^{Sc} has a high β-sheet content and less α-helical structure (Pan, K. M., et al. (1993) Proc. Natl. Acad. Sci. USA 90:10962-10966; Safar, J., et al. (1993) Protein Sci. 4:2206-2216. The sequence of PrP₁₀₆₋₁₂₆, KTNMKHMAGAAAAGAVVGGLG (SEQIDNO:1) is not only very hydrophobic, but at low pH it converts to a β-sheet conformation. Moreover, in solution, it can convert other prion peptides to β-sheet conformation.

Definitions

The terms “a”, “an” and “the” as used herein are defined to mean “one or more” and include the plural unless the context is inappropriate.

The terms “protein”, “peptide”, “polypeptide” and “oligopeptide” are used interchangeably herein and refer to chains of amino acids (typically L-amino acids) whose alpha carbons are linked through peptide bonds. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the protein, or to the alpha-amino group (imino group when participating in a peptide bond) of an amino acid at any other location within the protein. Similarly, the term “carboxy terminus” (abbreviated C-terminus) refers to the free carboxyl group on the amino acid at the carboxy terminus of a protein, or to the carboxyl group of an amino acid at any other location within the protein.

As used herein, reference to a “compound” is a reference to one or more such compounds and includes equivalents thereof known to those skilled in the art, and so forth. Unless defined otherwise, all technical and scientific terms used herein have the same meaning as commonly understood to one of ordinary skill in the art.

Synthetic Prion Molecules

Antigenic prion molecules, including proteins, peptides, protein fragments and peptide fragments were determined by testing synthetic PrP^{c} molecules for antigenicity. The synthetic molecules of PrP^{c} were created using methods known to those skilled in the art. For example, synthetic PrP^{c} peptides can be created based on published sequences. The sequences of the constructed synthetic peptides are then verified in situ by checking the sequences against available sequence data from databases including, but not limited to, GenBank by BLAST using the Vector NTI Advance package.

It was determined that synthetic fragment PrP^{c}₁₀₆₋₁₂₆ was the most neurotoxic fragment. The neuronal death induced by PrP^{c}₁₀₆₋₁₂₆ occurred by apoptosis in a dose dependent manner. In the terminal stages of subacute encephalopaties, such as scrapie, PrP^{Sc} reaches whole brain concentrations 10 to 20 times higher than PrP^{c} (Meyer, R. K. et al. (1986) Proc. Natl. Acad. Sci. USA, 83:2310-2314). The process of programmed cell death induced by PrP^{c}₁₀₆₋₁₂₆ is associated, among others, with the induction of the testosterone-repressed prostate message-2 gene (TRPM-2). It is not known whether apoptosis is activated in vivo during prion-related encephalopaties, but the expression of the TRPM-2 mRNA is increased 10-fold in scrapie-infected hamsters (Duguid, J. R., et al. (1989) Proc. Natl. Acad. Sci., USA 86:7620-7624).

While not wishing to be bound to any particular theory, it appears that a neurotoxic mechanism is possibly responsible for neuronal cell loss in prion-related encephalopathies and could also be relevant in Alzheimer's disease (Forloni, G., et al. (1992) Soc. Neurosci. Abs. 18:601-616).

Vaccines

A vaccine and methods for the prevention of neurodegenerative diseases are provided herein. A common problem in vaccination is that the administered vaccines are not sufficiently immunogenic, and not even repeated administration of the vaccine creates a protective immunity. To increase the immune response, immunostimulants or adjuvants are frequently admixed to vaccines. Such substances intensify or influence the immune response. Inorganic substances such as aluminum hydroxide as well as water-in-oil emulsions have been frequently used by scientists and are also useful in the vaccines described herein. Various cytokines such as interleukins, lymphokines or GM-CSF (granulocyte-macrophage colony-stimulating factor) have also been shown to have immunostimulating properties. For example, GM-CSF has been shown to stimulate the growth of dendritic cells and macrophages. Intradermal (ID) priming of the injection site each day for five days with GM-CSF has been shown to induce potent delayed type hypersensitivity responses (DTH) and antibody titers in rats. GM-CSF co-administration with antigen induced DTH and cytotoxic T lymphocytes (CTL) in human melanoma patients.

Preferably, the compositions provided herein contain at least one modified peptide, fragment or protein, preferably delivered in liposomal bilayers such as liposomes. The compositions are useful in methods to elicit an immune response. Preferably, these immune responses are able to overcome immune tolerance to “self” proteins. It is desirable to elicit such immune responses against prion peptides in order to treat or prevent neurodegenerative diseases. The products of the immune response are used to treat prion deposits. The products of the immune response, including but not limited to, antibodies, antisera, stimulated cells and cellular factors, and antigens of modified prion peptides, fragments or proteins, may also be included in the compositions described herein.

Preferred compositions contain peptides that are modified by the covalent binding of the peptides to moieties, such as lipophillic moieties. Such moieties are capable of presenting the peptide on the exterior of a delivery agent, such as by anchoring the peptide in the lipid wall of a liposome. Preferred methods include administration of the peptide-lipophillic moiety into subjects, such as humans, mammals or other animals, for example, by injection.

In a first preferrred embodiment, one or more prion peptide fragments are attached to liposomes to form lipopeptides. The following scheme will summarize the general approach to the solid phase synthesis of lipopeptides of the formula, wherein Xaa is the amino acid sequence of the peptide:

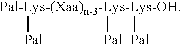

The Sheppard polyamide resin and Fmoc N(α) protection is considered to be advantageous in this case, although classical Merrifield resins and strategies are also possible.

TABLE 1
Synthetic chart for solid phase synthesis of membrane anchoring
lipopeptides.

| | |
|---|---|
| Carrier | e.g. HO—C₆H₄—OCH₂CONleNHCH₂CH₂NHCO-PA |
| N(α)-Protection | Fmoc |
| Side-chain | Boc, OBu, etc. |
| protection | |
| Attachment of | FmocLys(Pal)Pfp/DMAP/DMF/eventual |
| first and second | HOBT catalysis |
| residue | |
| N(α)- | 20% piperidine/DMF |
| Deprotection | |
| Coupling | FmocXaaOPfp/DMF, with catalysis by |
| | HOBT in some cases, or alternatively, |
| | (FmocXaa)₂O/DMF |
| Last coupling | Pal₂LysOPfp/DMAP/DMF/HOBT |
| Cleavage from | 95% aq. TFA |
| the carrier and | |
| side-chain | |
| deprotection | |
| Purification | Ion exchange chromatography, HPLC. |
| Analysis | Mass Spectrometry, Circular Dichroism |

In a second preferred embodiment, the composition contains at least one modified prion molecule such as a modified protein, a fragment of a modified prion protein, a modified prion peptide or a fragment of a modified prion peptide, wherein the protein, peptide or fragments is modified by covalently bonding lipophilic moieties to the protein, peptide or fragment. More preferably, the composition contains a modified prion PrP^{Sc}₁₀₆₋₁₂₆ peptide, wherein the modification is that the peptide includes covalently bonded lipophilic moieties. The protein, peptide or fragment may be anchored in a liposome or other liposomal bilayer or may be anchored in multilamellar vesicles. The protein, peptide or fragment is attached to the lipophilic moieties through compositions including, but not limited to, palmytolated amino acids, preferably, palmytoylated lysines, or by other methods known to those skilled in the art.

Liposomes

The formation and use of liposomes is generally known to those of skill in the art (as described by Couvreur, et al. (1977) FEBSLett. 84(2):323-326: Couvreur (1988) Crit. Rev. Ther. Drug Carrier Syst., 5:1-20; Lasic (1998) Trends Biotechnol., 16(7):307-321). Recently, liposomes were developed with improved serum stability and circulation half-times (as described by Abizon and Papahadjopoulos (1988) Proc. Natl. Acad. Sci. USA, 85:6949-6953; Allen and Choun (1987) FEBS Lett., 223:42-46; and U.S. Pat. No. 5,741,516). Liposomes have been used effectively to introduce genes, drugs (Heath and Martin (1986) Chem. Phys. Lipids, 40:347-358; Heath et al. (1986) Biochim. Biophys. Acta, 862:72-80; Balazsovits et al. (1989) Cancer Chemother. Pharmacol., 23:81-86; Fresat and Puglisi (1996) J. Drug Target, 4(2):95-101), radiotherapeutic agents (Pikul et al. (1987) Arch. Surg., 122(12):1417-1420;), enzymes (Imaizumi et al. (1990) Stroke, 21(9):1312-1317; Imaizumi et al. (1990) Acta. Neurochirurgica Suppl. 51:236-238), viruses (Faller and Baltimore (1984) J. Virol., 49(1):269-272;), transcription factors and allosteric effectors (Nicolau and Gersonde (1979) Naturwissenschaften (Gerrnany), 66(11):563-566) into a variety of cultured cell lines and animals.

Liposomes are formed from phospholipids that are dispersed in an aqueous medium and spontaneously form multilamellar concentric bilayer vesicles (also termed multilamellar vesicles). Multilamellar vesicles generally have diameters of from 25 nm to 4 μm. Sonication of multilamellar vesicles results in the formation of small unilamellar vesicles with diameters in the range of 200 to 500 Å, containing an aqueous solution in the core. The physical characteristics of liposomes depend on pH, ionic strength and the presence of divalent cations.

The ability to trap solutes varies between different types of liposomes. For example, multilamellar vesicles are moderately efficient at trapping solutes, but small unilamellar vesicles are extremely inefficient. Small unilamellar vesicles offer the advantage of homogeneity and reproducibility in size distribution. However, large unilamellar vesicles offer a compromise between size and trapping efficiency. These are prepared by ether evaporation and are three to four times more efficient at solute entrapment than multilamellar vesicles.

While not wishing to be bound by any particular theory, it is currently believed that liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents.

Administration of Vaccines

The compositions are useful as vaccines or in methods for eliciting a systemic, immunogen-specific immune response in a mammal by administering the composition to an animal, particularly a mammal. While any suitable carrier known to those of ordinary skill in the art may be employed in the vaccine composition, the type of carrier will vary depending on the mode of administration. Compositions of the present invention may be formulated for any appropriate manner of administration, including for example, topical, oral, nasal, intravenous, intracranial, intraperitoneal, subcutaneous, intradermal, intramuscular, transdermal, intraarticular, intracranial or intraspinal administration. The preferred route of administration is intravenous injection.

For parenteral administration, such as subcutaneous injection, the carrier preferably contains water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the compositions described herein. Alternatively, compositions may be formulated as a lyophilizate.

Effective dosages and administration methods for delivery of the compositions containing vaccines or antisera may be determined empirically and such determinations are within the skill of an artisan. Those skilled in the art will understand that the dosage required depends on the subject receiving the protein, the route of administration, the particular type of peptide antigen used and other substances being administered, among other considerations.

A suitable single dose size is a dose that is capable of eliciting an immune response in an animal when administered one or more times over a suitable time period. A single dose of a vaccine is an amount sufficient to reduce, eliminate, or prevent at least one symptom of the prion associated disease or elicit an immune response against the prion or the amyloid depositions caused by the accumulations of the prion.

In a preferred embodiment, an appropriate single dose of the amino acid:liposome portion of the composition described herein is from about 0.1 μg to about 100 μg per kg body weight of the mammal to which the complex is administered. In another embodiment, an appropriate single dose is from about 1 μg to about 10 μg per kg body weight. In another embodiment, an appropriate single dose of amino acid:liposome complex is at least about 0.1 μg of amino acid to the animal, more preferably at least about 1 μg of nucleic acid, even more preferably at least about 50 μg of amino acid, even more preferably at least about 100 μg of nucleic acid to the mammal.

A preferred number of doses of the vaccine described herein is from about 1 to about 10 administrations per patient, more preferably from about three to about eight administrations per patient, and more preferably from about three to about seven administrations per patient. Preferably such administrations are given once every two to four weeks, until signs of a therapeutic improvement appear, or until sufficient memory immune response is established to be considered effective for prevention of the disease or condition. Variation of the dose and frequency of administration can be determined by those of skill in the art.

The vaccine or antisera compositions may also be administered to the subject animal in combination with effective amounts of one or more other therapeutic agents. They may be administered sequentially or concurrently with the one or more other therapeutic agents. The amounts of vaccine or antisera compositions and therapeutic agent depend on the type of therapeutic agents are used, the condition being treated, and the scheduling and routes of administration, among other considerations. Following administration of vaccine or antisera compositions to the subject animal, the animal's physiological condition is monitored in various ways well known to the skilled practitioner.

In the methods described herein, vaccines and therapeutic compositions can be administered to any member of the vertebrate class of Mammalia, including, without limitation, primates, rodents, livestock and domestic pets. Livestock include mammals to be consumed or that produce useful products. Preferred mammals include humans, sheep, cattle, horses and pigs.

All publications and patents mentioned herein are incorporated by reference in their entireties for the purpose of describing and disclosing, for example, the constructs and methodologies that are described in the publications, which might be used in connection with the presently described invention.

The above disclosure generally describes the present invention. A more complete understanding can be obtained by reference to the following examples. These examples are described solely for purposes of illustration and are not intended to limit the scope of the invention. Although specific terms have been employed herein, such terms are intended in a descriptive sense and not for purposes of limitation.

### EXAMPLES

### Example 1

### Creation of Synthetic Peptides

Seven synthetic peptides, homologous to different segments of PrP^{c} are used to investigate their influence on the viability of primary rat hippocampal neurons. The sequences are set forth below in Table 2.

TABLE 2
Amino acid Secqences of Synthetic Peptides

| | | SEQ |
|---|---|---|
| Amino Acid Fragment | Sequence | ID NO |
| PrP 57-64 | WGQPHGGG | 2 |
| PrP 89-106 | WGQGGGTHSQWNKPSKPK | 3 |
| PRP 106-114 | KTNMKHMAG | 4 |
| PrP 106-126 | KTNMKHMAGAAAAGAVVGGLG | 1 |
| PRP 127-135 | GYMLGSAMS | 5 |
| PrP 127-147 | GYMLGSAMSRPIIHFGSDYED | 6 |
| PrP 106-126 | NGAKALMGGHGATKVMVGAAA | 7 |
| (Scrambled) | | |

The peptides are synthesized using solid-phase chemistry. F-moc (9-fluroenilmethoxycarbonyl) was used as the protective group for aminic residues, and 1-hydroxybenzotriazole, 2-(1H-bwenzotriazol-1-yl)-1,1,3,3-tetramethyluronium hexafluorophosphate and N,N-dicyclohexyl-carbodiimide as activators of carboxylic residues. The crude peptides are purified by crystallization and are verified by analytical reversed phase HPLC and amino-acid sequencing.

### Example 2

### Determiination of the Most Reactive Segments of the PrP^{c}

Cultures were prepared from rat hippocampus and exposed either acutely or chronically to PrP peptides, or to a scrambled sequence of PrP^{c}₁₀₆₋₁₂₆ as a control. For acute experiments, the peptides were applied once at the time of plating. For chronic treatment, they were added every two days for ten days. On day ten, cell viability was evaluated and compared with cultures treated with vehicle only.

Neuronal death occurs from chronic exposure of primary rat hippocampal cultures to micromolar concentrations of a peptide corresponding to residues 106-126 of the amino-acid sequence deduced from human PrP^{c} cDNA, in a concentration dependent manner. The data are shown in Table 3.

TABLE 3
Chronic treatment of hippocampal neurons - 9 days
Chronic treatment of hippocampal neurons - 90 days

Cell death %

| Peptide | 20 μm | 80 μm |
|---|---|---|
| PrP 106-126 (SEQ ID NO:1) | 18 ± 8 | 100 ± 8 |
| PrP 57-64 (SEQ ID NO:2) | 0 ± 5 | 3 ± 4 |
| PrP 89-106 (SEQ ID NO:3) | 5 ± 2 | 2 ± 6 |
| PrP 106-114 (SEQ ID NO:4) | 0 ± 3 | 12 ±0 6 |
| PrP 127-135 (SEQ ID NO:5) | 3 ± 6 | 15 ± 9 |
| PrP 127-147 (SEQ ID NO:6) | 1 ± 7 | 18 ± 7 |
| PrP 106-126 scrambled (SEQ ID NO:7) | 3 ± 2 | 8 ± 3 |

The data are the mean ± s.e. of 6-10 determinations and are normalized to the toxic effect of PrP 106-126 (designated 100% response).

### Example 3

### Synthetic Strategy for Anchoring Lipopeptides in Liposomes

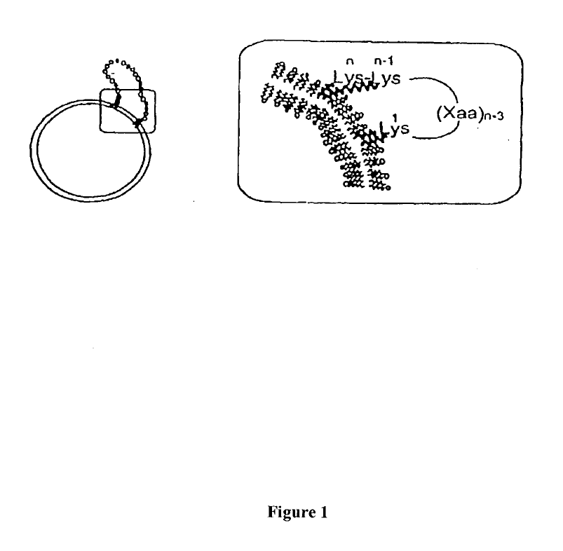

Lipopeptides that present an extraliposomal loop can be synthesized using current automated methods for solid phase peptide synthesis. The anchoring into the lipid mono or bilayer is ensured by long fatty acid chains, e.g. palmitoyl residues [CH₃(CH₂)₁₄CO—]. In order not to distort the synthetic membrane, two such residues must be present in close proximity at both ends of the desired peptide. Lysine is the ideal amino acid for inserting one palmitoyl residue at the ε-NH₂ or, alternatively, two palmitoyl residues at both the α- and ε-NH₂ groups. In the latter case, of course, this has to be the N-terminal amino acid. At the C-terminal, therefore, two ε-palmitoylamidolysines have to be inserted to ensure membrane anchoring. The “in-between” amino acids of the loop can be varied at will to ensure binding selectivity as well as other desired properties. (See FIG. 1)

Direct palmitoylation of Lys-(Xaa)ₙ₋₃-Lys-Lys-OH peptides, which could be accessed by routine solid phase synthesis, is impossible if the loop contains residues which could react with the palmitoylation reagent, i.e. unprotected amino acids such as Lys, Ser, Thy, Tyr, Cys, etc. Therefore the palmitoylated lysines are introduced during the solid phase synthesis as the following building blocks.

For the N-terminus:

H₃C—(CH₂)₁₄—CO—NH—(CH₂)₄—CH—CO-OPfp

H₃C—(CH₂)₁₄—CO—NH

Pal₂LysPfp (where Pfp represents the activated pentaflourophenol ester and Pal represents palmitoyl residues)

For the C-terminus:

Fmoc-NH—CH—CO-Opfp

FmocLys(Pal)Pfp (where Fmoc represents 9-fluorenylmethoxycarbonyl)

(CH₂)₄—NH—CO—(CH₂)₁₄—CH₃

Standard synthetic procedures for making the pentafluorophetiol ester (DCC/DMF) are combined with the literature described synthesis of Pal₂Lys-OH (Paquet, A.. (1976) Can. J. Chem. 54:733-737). Mono and dipulmitoyl-(L)-lysine have also been studied in crystallization studies (Landau, F. M. et al. (1989) J. Am. Chem. Soc. 111:1436-1445; Popovitz-Biro, R. et al. (1990), J. Am. Chem. Soc. 112:2498-2506). Furthermore Fmoc-Lys(Pal)-OH, the doubly protected amino acid required for the C-terminus, is commercially available (Baehem Bioscietices, King of Prussia, Pa.).

### Example 4

### Anchoring of FmocLys(PAL) on the Alkoxybenzyl Alcohol Resin

FmocLys(Pal)OH (from BACHEM) is reacted with the alkoxybenzyl alcohol resin (from BACHEM) in the presence of dicyclohexylcarbodiimide (DCC, Aldrich) and dimethyl-aminopyridine (DMAP, Aldrich) in dry, freshly distilled methylene chloride according to the procedure optimized by G. Lu et al. (1981) J. Org. Chem., 46, 3433. After stirring for three hours at room temperature, the reaction mixture is filtered and washed thoroughly ten times with dry methylene chloride. To ensure complete reaction, the obtained resin is reacted once more with a fresh portion of FmocLys(Pal)OH, in the presence of DCC and DMAP in dry methylene chloride at room temperature overnight. The next day, after filtration and washing with methylene chloride the resin is dried in vacuum. The palmitoylation is verified by FT-IR.

### Example 5

### Solid Phase Synthesis

The second palmitoylated lysine is added to the resin in Example 4 by means of FmocLys(Pal)OH after deprotection (removal of the Fmoc group). Then 16 cycles of synthesis are performed for the prion peptide PrP^{c}₁₀₆₋₁₂₆. For test purposes, a small quantity of this peptide is then cleaved from the resin and investigated by electrospray mass spectrometry (ES-MS).

The compound αε-dipalmitoyllysine is coupled to the remainder of the uncleaved resin. Even prolonged coupling times, i.e. overnight at room temperature, leave unreacted material (ninhydrin test) due to the low solubility in DMF of the dipalmitoylated lysine. After cleavage from the resin, ES-MS is used to determine that the desired tetrapalmitoylpeptide is present.

In a second run, to avoid the sluggish coupling with αε-dipalmitoyllysine, after the 16 cycles which attached the PrP^{c}₁₀₆₋₁₂₆ residues to the first two palmitoyllysines are completed, two sequential palmitoyllisines are inserted at the end, the coupling being performed twice for each and a sample is analyzed using ES-MS to verify the palmitoylation.

### Example 6

### Preparation of αε-dipalmitoyllysine

Direct palmitoylation or lysine with palmitoyl chloride in a Schotten-Baumann reaction with aqueous sodium hydroxide, on a 20 g scale leads to a material which contains appreciable amounts of palmitic acid and which could not be separated from the desired αε-dipalmitoyllysine. An indirect method is therefore used following the procedures of H. Kiwada, et al., Chem. Pharm. Bull., 35, 2935-39 (1987); Y. Lapidat, et al., J. Lipid Res., 9, 142-44 (1967).

The palmitoylester of N-hydroxysuccinimide is synthesized first from palmitic acid (Fluka), N-hydroxysuccinimide (Aldrich) in the presence of DCC (Aldrich) in ethyl acetate in 77% yield. This activated ester is subsequently reacted with the sodium salt of lysine in aqueous tetrahydrofurane. The crude product obtained after filtration and washing with water is analyzed using proton NMR and Fast Atom Bombardment to check for unreacted activated ester.

### Example 7

### Reconstitution of the Palmitoylated Peptides in Liposomes

Liposomes with Lipid A are used as adjuvants. They are prepared by mixing dimyristoyl-phosphatidylcholine, dimyristoylphosphatidyl-glycerol, and cholesterol (Avanti Polar Lipids, Alabaster, Ala., USA) in the molar ratios 0.9,0.1:0.7. Monophosphoryi lipid A, a strong immunomodulator, (IASL Biologicals, Campbell, Calif., USA) is added at a concentration of 40 mg per mmole of phospholipids. Tosi et al., (1995) Biochem. Biophys. Rcs. Comm., 212:494-500. The palmitoylated peptides are added at a molar ratio to phospholipids of 1:100 and 1:200. Solvents are evaporated. The resultant film, after hydration with sterile phosphate buffer saline (PBS, pH 7.4) with a final phospholipid concentration 4 mM, is further homogenized by orbital shaking. The liposome suspension is mixed with sterile Alum 15 minutes before injection (9:1 vol:vol, Rehydrogel, HYA, Rebeis Inc, Berkley Heights, N.J.).

### Example 8

### Manufacture of a Liposomal Prion Vaccine

Liposomes are manufactured as described (Alving, Schichijo, and Mattsby-Baltzer, Liposome Technology, vol. 2, pp. 157-175 (1984)) under sterile conditions. Dintyristoyl phosphatidylcholine, dimistoyl phosphatidylglycerol, cholesterol is in a molar ratio of 9:1:7.5, respectively. Lipid A is added to give a final product concentration of 400 μg/ml. Lipids are dried with chloroform by rotary evaporation. The dried lipid film is hydrated and lyophilized. Peptide antigen is added to the lyophilized lipids and incubated at 4° C. The liposomes are diluted in PBS, pll 7.4 and centrifuged at 30,000×g for 30 min to remove unencapsulated antigen. The supernatant is discarded and the pellet washed again. The final pellet is resuspended in PBS to give a final phospholipid concentration of 100 mM. Peptide antigen was at concentration of approximately 200 μg/ml.

### Example 9

### Immunization Protocol

Mice are immunized with 50 μl of peptide antigen-encapsulated liposomes and are boosted at week 4. Three different vaccine strategies are tested:

1. Peptide antigen-encapsulated liposomes administered by the intravenous route (IV).

2. Peptide antigen-encapsulated liposomes with GM-CSF injected at the same site of immunization.

The mouse abdomen is shaved and treated with NAIR three days prior to the start of GM-CSF injection. Ten μg of GM-CSF in 50 μl is injected ID each day for five days at the same site. On the day 5, 50 μl of peptide antigen-encapsulated liposomes are injected at the same site as the GM-CSF. The same protocol will be undertaken for the boost.

3. Oil-in-water emulsion containing peptide antigen-encapsulated liposomes.

As part of a cooperative research and development agreement between WRAIR-membrane Biochemistry and Jenner Biotherapies, an oil-in-water emulsion containing liposomes has been developed. The emulsion induced high titer antibody, DTH and potent lymphocyte proliferation responses in clinical trials with prostate cancer patients. In this study, GM-CSF is combined with the emulsion formulation. GM-CSF is administered as described above. The emulsion is formulated by emulsifying 1 ml of liposomes with 0.1 ml of light mineral oil. This is done by passing liposomes-mineral oil between 2 glass syringes connected with a 3-way stopcock at a rate of 2 passes per sec for 5 min. Fifty μl of the emulsion is injected ID.

The immunogenicity of the formulations is examined after two parenteral immunizations. Sera are analyzed for the presence of antigen-specific antibodies, and for the distribution of antibody isotypes. Spleen cells are used to evaluate cell-mediated immune responses. Those cells are tested for the presence of cytotoxic T lymphocytes (CTL) and lymphoproliferative (lymphoproliferation) cells.

### Example 10

### Assay for Cytotoxic T Lymphocytes

For CTL analysis, harvested cells from mice immunized as described above are cultured for 7 days in 6-well plates in the presence of 10 μg per ml of palmitoylated synthetic peptide SEQ ID NO:1. At the end of the culture period effector cells are assessed in duplicate for prion-specific cytolytic activity in standard [⁵¹Cr]-release assays using control and S-transfected P815 cells. Gp120-specific cytotoxicity is determined by using P815 target cells that are either left untreated or pulsed for 1 hr with peptide pCM1007. Minimum and maximum release are determined with target cells without effector cells and by the addition of 3% (v/v) Triton X-100, respectively. Results are expressed as percent [⁵¹Cr]-release (cpm of experimental culture—cpm of spontaneous release/cpm of maximum release—cpm of spontaneous release).

### Example 11

### Assay for Prion Specific Antibodies

Titration and isotyping of pooled sera is performed in a standard enzyme-linked immunosorbent assay (ELISA) format using plates coated with Prion Sera were diluted in PBS/BSA starting at 1:400. Biotinylated secondary antibodies specific for Ig or the isotypes IGg1, IgG2a and IgG2b followed by a horseradish peroxydase-streptavidin conjugate are used for detection of bound antibodies. ELISA titers are calculated from a reference by SoftmaxPro and expressed in ELISA units (EU/ml). Gp120-specific antibody titers are determined in a standard ELISA using plates coated with gp120 protein. Sera are diluted in PBS/Tween20/BSA starting at 1:100. Biotinylated secondary antibodies specific for Ig or the isotypes IgG1, IgG2a and IgG2b followed by a horseradish peroxydase-streptavidin conjugate are used for detection of bound antibodies. Titers are calculated in relative to a standard mouse Ig and expressed as μg/ml.

### Example 12

### Challenging of Immunized Mice

Prnp^{0/0} mice, in which both alleles of the PrP gene is ablated, were immunized with the PrP^{c}₁₀₆₋₁₂₆ lipopeptide. Once the immune response is positive and CTL are detected, the mice are challenged with 50 μl of brain extract from scrapie mice by injection and monitored for appearance of the disease.

What is claimed is:

1. A composition comprising at least one modified prion molecule, wherein the prion molecule is a prion protein, fragment of a prion protein, prion peptide, or fragment of a prion peptide, and wherein the modification comprises at least one covalently bonded lipophilic moiety.

2. The composition of claim 1, wherein the modified prion molecule is anchored in a liposomal bilayer.

3. The composition of claim 2, wherein the liposomal bilayer is a liposome.

4. The composition of claim 1, wherein the lipophilic moiety is a multilamellar vesicle.

5. The composition of claim 1, wherein the molecule is covalently bonded to the lipophilic moiety by a palmitoylated amino acid.

6. The composition of claim 1, wherein the amino acid is lysine.

7. The composition of claim 1, further comprising a carrier or therapeutic agent.

8. The composition of claim 7, wherein the therapeutic agent is a cytokine.

9. The composition of claim 1, wherein the molecule is a PrP^{c}₁₀₆₋₁₂₆ peptide.

10. The composition of claim 1, wherein the molecule is antigenic.

11. A method for eliciting an immune response in an animal, comprising administering to the animal a composition comprising at least one modified prion molecule, wherein the prion molecule is a prion protein, fragment of a prion protein, prion peptide, or fragment of a prion peptide, and wherein the modification comprises at least one covalently bonded lipophilic moiety.

12. The method of claim 11, wherein administration of the composition to the mammal produces immunization against prion diseases or stimulation of effector cell immunity against prion diseases or conditions.

13. The method of claim 12, wherein the diseases are central nervous system spongiform encephalopothics.

14. The method of claim 13 wherein the encephalopthies are scrapie, transmissible mink encephalopathy, chronic wasting disease, bovine spongiform encephalopathy, Creutzfeldt-Jacob disease, Gerstmann-Strussler-Scheinker syndrome, fatal familial insomnia, kuru or alpers syndrome.

15. The method of claim 11 wherein the modified prion molecule is anchored in a liposomal bilayer.

16. The method of claim 11 wherein the molecule is covalently bonded to the lipophilic moiety by a palmitoylated amino acid.

17. The method of claim 11 wherein the molecule is a PrP^{c}₁₀₆₋₁₂₆ peptide.

18. The method of claim 11, wherein the molecule is antigenic.


SEQUENCE LISTING (7 sequences)

SEQ ID NO: 1 - protein, 21 aa, Artificial Sequence
  Synthetic
KTNMKHMAGAAAAGAVVGGLG

SEQ ID NO: 2 - protein, 8 aa, Artificial Sequence
  Synthetic
WGQPHGGG

SEQ ID NO: 3 - protein, 18 aa, Artificial Sequence
  Synthetic
WGQGGGTHSQWNKPSKPK

SEQ ID NO: 4 - protein, 9 aa, Artificial Sequence
  Synthetic
KTNMKHMAG

SEQ ID NO: 5 - protein, 9 aa, Artificial Sequence
  Synthetic
GYMLGSAMS

SEQ ID NO: 6 - protein, 21 aa, Artificial Sequence
  Synthetic
GYMLGSAMSRPIIHFGSDYED

SEQ ID NO: 7 - protein, 21 aa, Artificial Sequence
  Synthetic
NGAKALMGGHGATKVMVGAAA